(19) United States
(12) Patent Application Publication
(10) Pub. No.: US 2026/0207074 A1
(43) Pub. Date: Jul. 23, 2026

(54) Breath Analyzer Device

(71) Applicants: Uday Singh, Stewartsville, NJ (US); Tejas Singh, Stewartsville, NJ (US); Neha Singh, Stewartsville, NJ (US)

(72) Inventors: Uday Singh, Stewartsville, NJ (US); Tejas Singh, Stewartsville, NJ (US); Neha Singh, Stewartsville, NJ (US)

(21) Appl. No.: 18/230,701

(22) Filed: Jan. 21, 2025

(51) Int. Cl. A61B 5/08; A61B 5/00; A61B 5/097; G01N 30/02

(52) U.S. Cl. CPC A61B 5/082; A61B 5/097; A61B 5/742; G01N 2030/025

(57) ABSTRACT

A system and device for analysing human breath and providing a perspective on whether the breath foul smells or is presentable. The system could be installed in homes or in public bathrooms (such as restaurants, airports), allowing its user to check their breath before going off to their business or social meetings with others. The system is provided with connectivity to cloud server comprising the relevant reference data, one or more sensors configured to identifying the components of the breath (that will be blown into the system) and by using the combination of reference data that is stored on the Cloud Server as well as the sensory data from one or more of its sensors, and generating the perspective on the breath.

### FIELD OF INVENTION

The present invention relates to an interactive breath analyser device which is able to be mounted in a public place is provided to be conveniently used by the public anytime. Moreover, this invention relates to an interactive breath analyser device which consists of a breath funnel, a canister, mini gas chromatographer, neural network module, embedded computer and LED lights.

### BACKGROUND

Communication between people is becoming important in modern society, and bad breath can be a big obstacle in communicating. Bad breath can be caused due to many reasons such as food residue, plaque, tongue coating, etc. In recent years, there has been a growing interest in maintaining good breath and towards that goal a wide variety of bad breath care products such as etiquette gum, mouthwash, deodorant and aromatic tablets have become available on the market. With such growing interest in identification and prevention of bad breath, a technique for simply determining at home whether there is a bad breath that causes discomfort to others is desired.

There exists tester to check the degree of drunk, detecting alcohol concentration on the display. JPH08145928A is an inside vehicle drinking checker that allows to easily check the person's alcohol degree while driving a car.

U.S. Pat. No. 4,823,803A is a type of halitosis detector device for detecting human halitosis. The device has an air inlet through which the exhalation to be tested is admitted into the chamber and an air outlet through which the exhalation tested is discharged from the chamber.

However, these devices are applicable for specific use such as halitosis or measuring alcohol level. Thus, there is a dire need for a checker device which can be installed at home or office or any other public place so the user can check whether he/she has bad breath or not before stepping out in public. It would be greatly helpful if the users were able to check their breath before meetings or dates, while at office or after a meal. The device has to be an affordable solution with a rapid indicator for testing bad breath.

### SUMMARY OF THE INVENTION

The present invention is a method and device for breath testing unit to provide a perspective on whether the breath is foul smelling or presentable. Moreover, the invention is intended to help its user with assessing whether they have bad breath or not along with achieving that objective, this invention will be an easy to install unit, also referred to as plug and play.

An object of the present invention is to provide a bad breath check method and a bad breath check unit that can be objectively and reliably determined, have a compact shape that is optimal for home use, and is economical.

The present invention achieves the objective by leveraging mini gas chromatograph as an indicator and neural networks based algorithms to detect whether the breath sample is acceptable or not. This invention is a small unit which can be fitted in any room and includes a breath funnel, mini gas chromatograph, embedded computer and a neural networks module.

The breath funnel placed on the top of the unit in which the user is required to blow into the funnel that will direct the breath sample into a small canister, where the breath sample will be further analysed for its components. In the next step, the mini gas chromatograph analyses the breath sample from the canister and outputs into several known Volatile Organic Compounds (VOCs). Further, the embedded computer on the unit will process the output of the mini gas chromatograph and uses the neural network module to assess whether the breath is acceptable or not and accordingly lights up the appropriate LED. The neural networks module leverages the pre-trained data set containing the volatile organic compounds (VOCs) composition across thousands of breath sample to process the current breath sample.

In order to test his or her breath, the user breathes or blows into the funnel on the device, and the sample is analysed via mini gas chromatograph based on pre-determined VOCs, whose readings are then passed to the neural networks module to assess the purity of the breath and according LED is lit on the unit that indicates that the user about his/her breath. This breath testing device therefore alerts the user of his or her bad breath and allows them to take appropriate action, such as to brush his or her teeth, use mouthwash or eat a breath mint.

### BRIEF DESCRIPTION OF DRAWINGS

FIG. 1 represents the various hardware components of the present invention.

FIG. 2 graphs out the software components of the current invention.

FIG. 3 lays out the decision-making framework that works inside the invention.

FIG. 4 represents one potential configuration in which the present invention might be mounted in a public restroom or bathroom in a commercial establishment.

### DETAILED DESCRIPTION OF THE INVENTION

Various embodiments of the disclosure are discussed in detail below. While specific implementations are discussed, it should be understood that this is done for illustration purposes only. A person skilled in the relevant art will recognize that other components and configurations may be used without departing from the scope of the disclosure.

The present disclosure overcomes one or more shortcomings of the prior art and provide additional advantages discussed throughout the present disclosure. Additional features and advantages are realized through the techniques of the present disclosure. Other embodiments and aspects of the disclosure are described in detail herein and are considered a part of the claimed disclosure.

According to one embodiment of the invention, a breath analyser device is computed within a unit which consists a breath funnel, a canister, a mini gas chromatographer, a neural network module and an embedded computer. Moreover, the breath funnel in the unit is used by the user to blow into it that will direct the breath sample into a small canister (also known as column), where the blown breath sample will be further analysed for its components. Another component of the invention is a mini gas chromatograph (MGC) which analyses the breath sample in the canister and outputs the breath into various known volatile compounds (VOCs). In addition, the neural networks module uses the pre-trained machine learning model which is prepared from data set containing the volatile compounds (VOCs) composition across thousands of breath samples to process the current breath sample.

In one of the embodiments, the volatile compounds (VOCs) are emitted as emitted as gases from certain solids or liquids. The human body can generate VOCs from both breath and whole-body skin.

The breath analyser includes a simple supporting member, such as a substantially transparent tube or straw containing the breath funnel from where the breath sample is passed onto the canister, present inside the Mini gas chromatographer (MGC). According to one of the embodiment, MGC is a mini version of gas chromatographer. Gas chromatography is an analytical technique used to separate and detect the chemical components of a sample mixture to determine their presence or absence and/or quantities. In the Mini gas chromatographer (MGC), the sample of the breath is entered into the canister (column) where the breath constituents are separated into various VOCs (Volatile Compounds). The separated VOCs are then analysed further based on the data available from neural networks module to identify and output a quality of the breath based on this determined level.

In one embodiment, the present invention can be installed but not limited to, in bathroom of a residence.

In yet another embodiment, the invention can be installed as part of vending machine that will analyse breath once the user make payment via cash or other payment modes such as credit card or GooglePay.

According to another embodiment, the breath analyser device can be powered solely by any external power source such as but not limited to an electrical outlet in the room. Furthermore, the present invention can be powered by both an external power source as well as built-in battery packaged with this invention during short-term power supply needs.

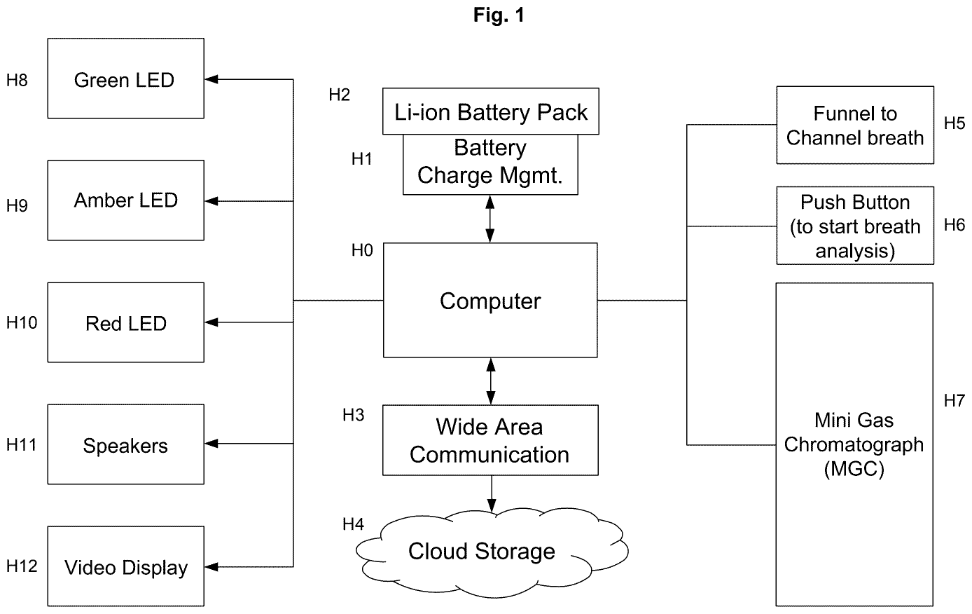

FIG. 1 represents the various hardware components of the present invention where the components can be grouped into 5 categories as listed: (A) Computer and data storage—Component H0 (computer) houses the main computing unit (CPU), graphical processing unit (GPU), local storage data storage (including RAM and solid-state storage), short-range wireless connectivity (Bluetooth), and local area network (ethernet and Wi-Fi). The next component H4 (Cloud storage) provides long-term and high-availability storage for data (VOCs composition of various breath samples) generated by this invention. In one embodiment, the computing unit which is coupled with data storage comprises an algorithm that when executed, the computing unit determined the level of known Volatile Compounds (VOCs) by the MGC. In an embodiment, the samples data thus stored will be used for further training of the AI agent.

Another category of the device is (B) Sensors—component H5 (funnel to channel breath) provides a place for the user to exhale or blow their breath into and it will channel the breath into a canister which is placed inside the mini gas chromatographer (MGC). In addition, component H6 (Push button) triggers the invention to get ready to accept the breath and start the analysis. Moreover, component H7 (Mini gas chromatographer) is a mini version of the gas chromatographer that will analyse the breath sample and output the composition of the breath into several known volatile compounds (VOCs).

In the next category, output hardware is shown which consists of LED as visual indicators and speakers. Component H8 (Green LED) is turned on when the breath analyser device has detected appropriate breath i.e. “acceptable”. Component H9 (Amber LED) is turned on when the detected breath is moderately “acceptable”. Component H10 (Red LED) is turned on when the detected breath is not considered “acceptable”. One another component is present H11 (Speakers) which will sound out more detailed messages with each turned on LED.

Additionally, another category is (D) Connectivity—Component H3 (Cellular modem) is present as an optional embodiment which includes the provision to insert cellular modem capable of 3G/4G/5G communication and an activated SIM card. This component further includes both the Ethernet and Wi-Fi adapters.

Category (E)—Component H1 (Battery Charge Management) is an optional embodiment, which provides the invention the ability to charge its battery pack and to monitor the amount of remaining charge in the battery pack. Component H2 (Li-ion Battery Pack) provides its own power in need of short-term power supply during emergency.

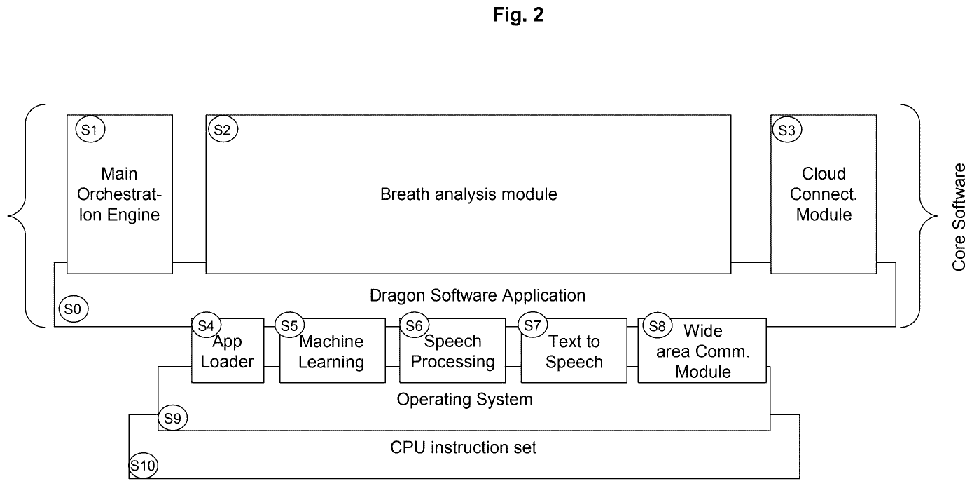

FIG. 2 graphs out the various software components of the invention which is further grouped into 2 categories: (A) Core Software—Component S0 (Dragon Software Application) is the software module that monitors the health of the various hardware and software components of the present invention. Component S1 (Main Orchestration Engine) is the software module that coordinates the different inputs (e.g., push button being pressed, breath being blown into the funnel) and processes the output of the breath analysis module. The next component S2 (Breath Analysis Module) is the software module that has a high degree of complexity as it leverages artificial intelligence (AI) based neural network algorithms to identify when different dishes of the palate. Component S3 (Cloud Connect Module) is the module that contains the software logic to connect to the cloud service and manages cloud storage for the present invention.

In this figure, the another category is (B) Supporting Software—Component S4 (App Loader) is the software module that will launch the S0 to S3 modules. Component S5 (Machine Learning) is the module that provides all the supporting infrastructure required for the breath assessment including machine learning to be able to detect whether the breath is acceptable or not. S6 (Speech Processing) is an optional component of the invention as speech processing module. Component S7 (Text to Speech Processing) is the module of this category that will convert any software generated messages into human understandable sounds that the device will play over the speakers (H11). The next component S8 (Wide Area Communication Module) has the software logic to manage the wide area network hardware (H3). S9 (Operating System) is the software module that manages the CPU, GPU, display, communication network, and peripherals (including inputs and outputs). The last component of this category S10 (CPU Instruction Set) is the foundational instruction set that the CPU of the computer (H0) provides upon which the operating system (S9) and the dragon application (S0) provides are executed.

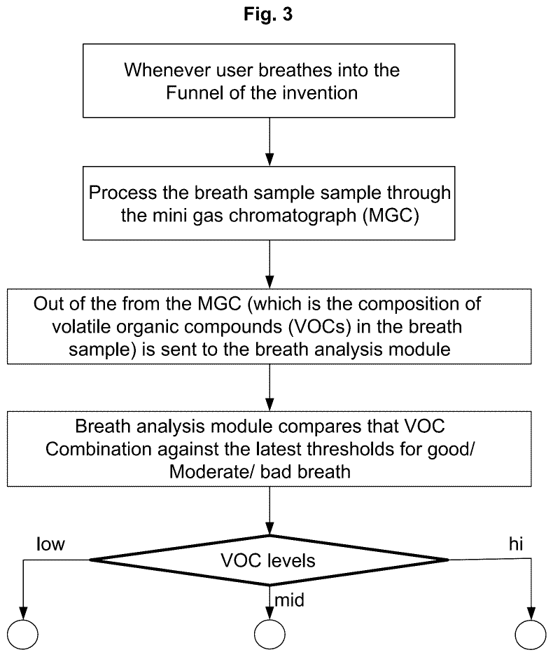

FIG. 3 lays out the decision making framework for the work inside the device. The finer grained details such as but not limited to analysing the breath (e.g., temperature), applying intelligent filters (e.g., whether the breath is acceptable), and notifying the user.

The flowchart depicted in this figures explains the working of the device. It initiates when the user breathes into the funnel placed on the top of the device of the present invention. The sample breath then enters into the canister or column of the mini gas chromatographer (MGC) for analysis. From this analysis, the different VOCs (volatile compounds) composition in the breath is sent to the breath analysis module. This breath analysis module compares the VOCs of the sample against the latest thresholds for good/moderate/bad breath. Depending upon the result of this analysis, the LED on the front of the device is turned on as green/mid/red.

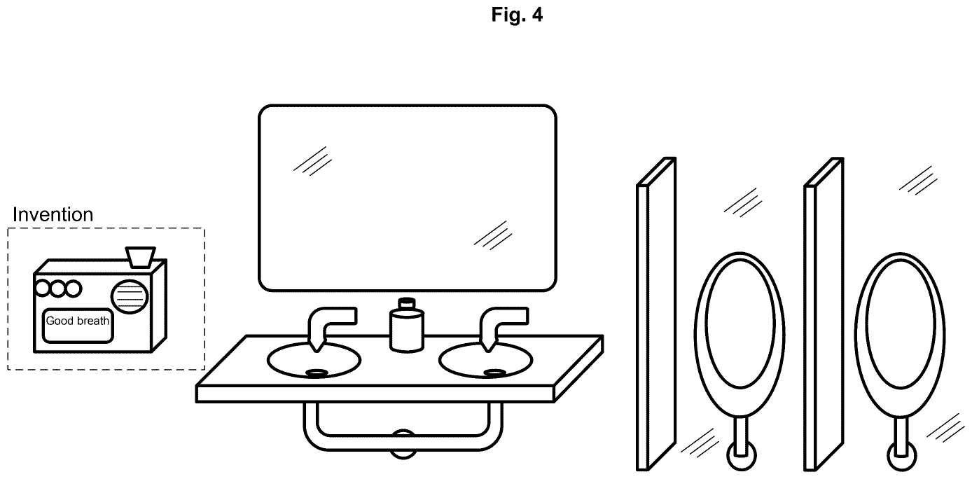

FIG. 4 simply shows an example of the placement of the device in a public bathroom. The invention unit is placed besides the sink of the bathroom.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A breath analyzer device, comprising:
a breath funnel to receive a breath sample from a user;
a mini gas chromatograph (MGC) coupled with the breath funnel through a canister, wherein the mini gas chromatograph is configured to
receive the breath sample of the user;
analyze the received breath sample; and
output the breath into known volatile compounds (VOCs); and
a computing unit coupled with data storage, wherein the data storage comprises an algorithm that when executed by the computing unit causes the computing unit to,
determine a level of known volatile compounds from the outputted volatile compounds (VOCs) by the mini gas chromatograph (MGC); and
identify and output a quality of the breath based on the determined level of the known volatile compound.

2. The breath analyzer device of claim 1, wherein the computing unit uses one or more output devices to output the quality of the breath.

3. The breath analyzer device of claim 1, wherein the one or more output devices include one or more LEDs, a display screen, or a speaker.

4. The breath analyzer device of claim 1, wherein the one or more LEDS are of different colors, each color is associated with a different level of quality of the breath.

5. The breath analyzer device of claim 1, wherein the mini gas chromatograph (MGC) comprises the canister to analyze the breath sample and identify the components of the breath sample.

6. The breath analyzer device of claim 5, wherein the mini gas chromatograph (MGC) is further configured to identify the known volatile compounds from the identified component of the breath sample.

7. The breath analyzer device of claim 1, wherein the computing unit comprises a neural network trained by a machine learning algorithm to identify the quality of the breadth of the user.

8. The breath analyzer device of claim 1, wherein the data storage comprises a dataset containing a plurality of volatile compound (VOCs) compositions detected from a plurality of breath samples to train the neural network.

9. The breath analyzer device of claim 1, wherein the device further comprises a wireless network module configured to couple the computing unit with a cloud storage over a wireless network, wherein the cloud storage stores a data comprising a plurality of VOCs compositions of various breath samples.

10. The breath analyzer device of claim 1, wherein the breath analyzer device is a compact device comprising a main housing for the configuration of components of the breath analyzer device.

11. The breath analyzer device of claim 10, wherein the housing comprises a top panel, a bottom panel and plurality of side panels defining the housing for the configuration of the components of the breath analyzer device.

12. A method of breath analysis, comprising:
receiving a breath sample from a user, using a breath funnel;
analyzing the received breath sample, using a mini gas chromatograph (MGC);
outputting the breath into known volatile compounds (VOCs), using the mini gas chromatograph (MGC); and
providing a computing unit coupled with data storage, wherein the data storage comprises an algorithm that when executed by the computing unit causes the computing unit to,
determine a level of known volatile compounds from the outputted volatile compounds (VOCs) by the mini gas chromatograph (MGC); and
identify and output a quality of the breath based on the determined level of the known volatile compound.

13. The method of claim 12, wherein the computing unit uses one or more output devices to output the quality of the breath.

14. The method of claim 12, wherein the one or more output devices include one or more LEDs.

15. The method of claim 12, wherein the one or more LEDs are of different colors, each color is associated with a different level of quality of the breath.

16. The method of claim 12, wherein the step to output the quality of the breath may further include steps of displaying the quality over a display screen or emitting a sound over speakers.
